(19) United States
(12) Patent Application Publication
(10) Pub. No.: US 2014/0255906 A1
(43) Pub. Date: Sep. 11, 2014

(54) ELECTROMAGNETIC BLOOD PRESERVATION AND STORAGE

(71) Applicant: Dan L. Dietz, Houston, TX (US)

(72) Inventor: Dan L. Dietz, Houston, TX (US)

(21) Appl. No.: 12/952,382

(22) Filed: Nov. 23, 2010

Related U.S. Application Data
(60) Provisional application No. 61/263,450, filed on Nov. 23, 2009.
(60) Provisional application No. 61/409,838, filed on Nov. 3, 2010.

(51) Int. Cl. A01N 1/02; C12M 3/00; C12M 1/00

(52) U.S. Cl. 435/2; 435/307.1; 435/283.1

(57) ABSTRACT

A method and apparatus for storing RBC wherein electromagnetic stimulation such as applying an electrical current or magnetic field to the stored blood extends the viability or shelf life of the stored RBC.

### CROSS REFERENCE TO RELATED APPLICATIONS

This application is a nonprovisional of U.S. 61/263,450 filed Nov. 23, 2009, a nonprovisional of U.S. 61/409,838 filed Nov. 3, 2010, and related to U.S. Ser. No. 12/433,566 filed Apr. 30, 2009.

### TECHNICAL FIELD

The present disclosure relates generally to blood preservation, storage and transfusion.

### BACKGROUND

Whole blood and blood components from a donor are commonly preserved and stored under refrigeration until they are required by a patient receiving the transfusion. Blood storage under refrigeration generally depletes the metabolites used by the blood during circulation in the body to maintain red blood cell (RBC) viability and function, and at the same time generates waste products that would otherwise be removed in the body. Sterile solutions containing anticoagulant and/or preservative systems are generally used in an attempt to maintain RBC viability and decrease the possibility of bacterial contamination.

Alterations in RBC biochemistry and physical properties that occur during storage are generally referred to as “storage lesions.” Refrigeration slows but does not stop RBC metabolism, and RBCS in storage continue to metabolize glucose through the anaerobic glycolysis pathway, producing two adenosine diphosphates (ADPs, from adenosine triphosphate or ATP) and a lactic acid during the metabolism of each glucose to 2,3-biphosphoglycerate (BPG, also referred to as DPG) or 1-phosphoglycerate (PG). BPG, which is widely accepted as necessary for allostearic facilitation of oxygen release from RBC in the body, is favored by a higher pH, whereas a lower pH favors PG, which has no effect on the oxygen dissociation curve. The tendency for pH in stored RBC to drop over time is only partially inhibited by the buffering capacity of the preservative and additive solutions currently in use.

ATP levels also decline during RBC storage, depleting at the expiration date to only from 45 to 86 percent of the original levels, depending on the storage additives used. While low ATP levels are associated with poor RBC viability, a high ATP level does not necessarily indicate good viability because of other types of storage lesions. Sodium and potassium leak through the membranes of the RBCs, elevating the potassium levels in the storage solution. BPG levels, generally associated with pH, may stay almost normal during the first week of storage, but also decline to the expiration date. Decreased BPG levels are associated with a left-shift in the oxygen dissociation curve of hemoglobin, resulting in an inhibited ability to release oxygen in the tissues of the recipient until circulation restores normal BPG levels, which can take up to 24 hours after transfusion.

Also, plasma hemoglobin levels continually increase due to RBC hemolysis that continues during storage. Blood ammonia levels also increase during storage. Further, RBCs manifest physical changes during storage, including the appearance of RBCs called echinocytes, which have multiple spiny projections; or the appearance of spherocytes, which take on a spherical shape as opposed to the normal biconcave disc shape of a healthy RBC.

RBCs in the body generally last about 120 days before hemolysis. However, the shelf life of RBCs in the available storage protocols is at most 3 to 6 weeks. Furthermore, recent studies have suggested that morbidity and mortality statistically increase with the length of storage of the RBCs, i.e., their storage age, prior to transfusion, especially after 1-2 weeks in storage.

Oxyhemoglobin (oxyHb) prevalent in arterial blood is diamagnetic with a reported susceptibility of −((0.13 to 0.65)×10⁻⁸ cgs emu/cm³Oe; whereas deoxyhemoglobin (deoxyHb) which occurs predominantly in venous blood, following oxygen release in the capillaries, is paramagnetic with a reported susceptibility of +(13 to 33)×10⁻⁸ cgs emu/cm³Oe. Methemoglobin, (metHb) in which the heme is essentially irreversibly oxidized, is also paramagnetic with susceptibility similar to that of oxyHb. The effects of strong magnetic fields, e.g., 30 to 100 kG, on blood have been reported in the literature as including orientation of red blood cells and platelets with the magnetic field direction, polymerization and alignment of fibrinogens, and increasing the apparent viscosity of blood. Mayrovitz et al., “Effects of a static magnetic field of either polarity on skin microcirculation,” Microvascular Research, vol. 69, pp. 24-27 (2005), reported a reduction in skin blood perfusion upon exposure of the patient to a neodymium magnet with a surface field of more than 4 kG.

There remains a long-felt and dire need in the art to inhibit the degradation of stored blood and blood components, to lengthen the shelf life, to improve the viability of RBCs in storage, to reduce the occurrence of complications associated with transfusions, and/or to reduce morbidity and mortality outcomes in transfusion recipients.

### SUMMARY

An aspect of the present invention is the improvement of the viability and/or shelf life of stored red blood cells (RBC or RBCs) by electromagnetically treating the blood in storage, e.g., by continuous or periodic application of electromagnetic stimulation, such as an electrical current, magnetic field, or combination thereof.

Another aspect of the present invention is an apparatus for storing blood comprising an electromagnetic generator to continuously or periodically generate electromagnetic stimulation in a blood storage compartment and/or blood flow path, e.g., an electrical current, magnetic field or combination thereof.

The inventor has determined that the deterioration of RBC in storage, i.e., the period of time following collection from a donor until transfusion into a recipient patient, may arise at least in part from the extended period of electromagnetic inactivity or quiescence, which is termed “electromagnetic senescence” herein. This phenomenon might be explained as a gradual degaussing or loss of surface polarization of the RBC, or a loss of magnetization of the heme centers in the hemoglobins, and/or a redistribution of polarity, although the invention is not to be limited by any particular theory.

The RBC in venous blood collected for blood banking and eventual transfusion, containing some deoxyhemoglobin, has an external surface orientation or polarity that helps keep the blood cells from sticking together due to the mutual repulsion of the like surface polarity. As the blood travels through the circulatory system, it is constantly cycled through bioelectromagnetic processing parameters that keep the heme irons magnetized and reconditioned for readily holding and releasing oxygen in repeated cycles through the cardiovascular circulatory system.

In the tissue or organ capillaries outside the lungs, the RBC are forced in close proximity to the internal surfaces of the capillary, oxygen is released and carbon dioxide taken on. The cells forming the capillary comprise a single-cell layer, and have bioelectromagnetic activity with intracellular electrical potential reported to be as much as 3 million ev/m in human cells. The capillary cells, and possibly to a lesser extent the surrounding tissue cells, are thus capable of bioelectromagnetic stimulation of the magnetically susceptible RBC, in addition to the electrical current incidental to the cardiac cycle and other neural and/or muscular activity. This is consistent with the observation that oxygen is more readily released in the vicinity of active muscles and/or organs where it is needed most.

One theory formed by the inventor, by which the present invention including the claims are not to be limited, is that the bioelectromagnetic stimulation may induce the hemoglobin in the magnetically susceptible RBC to roll or turn so that the external polarity is switched from negative or north (diamagnetic) to positive or south (paramagnetic) to facilitate release of the oxygen in the tissues. In the vicinity of the lungs and heart, the cardiac cycle can be a source of the bioelectromagnetic stimulation of the magnetically susceptible RBC, as well as the capillary cells, which are thought to stimulate the magnetization of the heme irons to facilitate carbon dioxide release and oxygen absorption.

Once the blood is withdrawn from a vein and collected, however, the RBC in conventional collection and storage systems and methodologies are no longer subjected to the repetitive bioelectromagnetic stimulation experienced in normal circulation through the body. Thus, the magnetic and electrical properties of the RBC in storage can be gradually altered, and the hemoglobin observed to rapidly deteriorate and lose the ability to selectively bind and release oxygen and carbon dioxide.

In one embodiment of the invention, RBC in stored blood or blood components is continuously or periodically subjected to electromagnetic stimulation, preferably on the order of biological electromagnetic stimulation, such as, for example, an electrical current and/or electromagnetic fields, similar in magnitude and phase characteristics to those experienced in the body, to constantly rejuvenate the RBC and maintain heme iron magnetization and/or external-internal polarity. In an embodiment, the applied electromagnetic stimulation serves to maintain the heme iron magnetization and/or surface magnetic polarity of the RBC, inhibiting electromagnetic senescence, preserving the RBC and inhibiting deterioration of the RBC.

It is to be understood that both the foregoing general description and the following detailed description are exemplary and are not restrictive of the present disclosure, as claimed. In addition, structures and features described with respect to one embodiment can similarly be applied to other embodiments.

### BRIEF DESCRIPTION OF THE DRAWINGS

The accompanying drawings, which are incorporated in and constitute a part of this specification, provide illustrative embodiments of the present disclosure and, together with the description, serve to explain the principles of the disclosure.

FIG. 1 schematically illustrates a blood storage and electromagnetic charge stimulation of blood cells according to an embodiment of the invention.

FIG. 2 is a side view of the blood storage compartment shown in FIG. 1.

FIG. 3 schematically shows a side section of tubing blood storage compartment including a source of electromagnetic fields for magnetically stimulating the blood during storage according to an embodiment.

FIG. 4 schematically shows a blood storage system with a blood circulation circuit for treating the blood during storage according to an embodiment.

FIG. 5 shows a red blood cell passing through a capillary tube with an externally opposing magnetic surface field according to an embodiment.

FIG. 6 shows a red blood cell passing through a capillary tube with an externally opposing magnetic surface field according to an alternate embodiment.

### DETAILED DESCRIPTION

In an embodiment, a method of storing red blood cells (RBC) comprises electromagnetically stimulating the RBC to improve viability.

In an embodiment, the RBC storage method comprises periodically or continuously passing an electric current through the stored RBC. In an embodiment, the electric current has amperage, voltage, wave form or a combination thereof corresponding to a cardiac cycle for the species of RBC. In embodiments, the current is direct current or alternating current. In an embodiment, the current is pulsed at a frequency from 0.001 to 10 Hz. In an embodiment, the current is supplied at a voltage potential between from 1 to 1000 millivolts.

In another embodiment, the RBC storage method comprises periodically or continuously applying a magnetic field to the RBC. In embodiments, the magnetic field comprises a static magnetic field, or an oscillating magnetic field, and the magnetic field can be a homogenous magnetic field or a heterogeneous magnetic field. In an embodiment, the magnetic field is within a range of from about 0.5 to about 500 Gauss, or within a range of from about 10 to about 100 Gauss. In an embodiment, the magnetic field is pulsed at a frequency from 0.001 to 10 Hz.

In one embodiment, the electromagnetic stimulation is applied to the RBC just prior to or during transfusion into the recipient.

In an embodiment, the RBC can be stored in the presence of an added anticoagulant, pH buffer, nutrient, preservative, pathogen inactivator or combination thereof.

In an embodiment, the RBC are stored in whole blood. The RBC can be stored, for example, in the presence of citrate-potassium-dextrose solution (CPD) such as CPD-1 or citrate-potassium-dextrose-adenine solution (CPDA) such as CPDA-1.

In another embodiment, the RBC can be separated from whole blood, e.g. by centrifugation or aphoresis. In an embodiment, the RBC can be stored in the presence of adenine-saline solution (AS), e.g., AS-1, AS-2, AS-3, AS-4, AS-5, AS-6, and so on.

In an embodiment, the method can include the step of inactivating pathogens, e.g., viruses, bacteria, parasites and so on, such as for example by adding a pathogen inactivator, such as in the Cerus INTERCEPT blood system, to the storage medium. Pathogen inactivators and inactivation methods are disclosed in U.S. Pat. No. 7,611,831, U.S. Pat. No. 7,293,985, US 2004/029897, US 2003/082510, US 2003/113704, U.S. Pat. No. 6,951,713, U.S. Pat. No. 6,709,810, WO 0191775, and U.S. Pat. No. 6,420,570, which are hereby fully incorporated by reference.

In an embodiment, the RBC can be processed using techniques well known in the art, e.g., the storage method can include contacting the RBC with a rejuvenation solution, such as pyruvate-inosine-phosphate-adenine solution (PIPA), irradiating the RBC, and the like.

In an embodiment, the RBC storage method can include gas exchanging the RBC to add or remove oxygen, carbon dioxide or a combination thereof. Additionally or alternatively, the RBC storage method can include dialysis to remove waste products from the RBC.

In an embodiment, the method may include agitating a medium such as plasma comprising the RBC, for example, pumping the medium comprising the RBC. In an embodiment, the RBC medium is passed through an electromagnetic stimulation zone.

The RBC storage method in embodiments can include controlling the temperature of the RBC between 1 and 6° C., or between about 30 and about 40° C.

The RBC storage method in one embodiment can be used to store the RBC in whole blood, optionally with an additive, for a period of time in excess of 35 days. The RBC storage method in another embodiment is used to store the RBC for a period of time in excess of 45 days. In other embodiments, the RBC storage method is effective such that the RBC and/or the storage media comprise more than 84% viable cells (as determined 24 hours post transfusion) following storage for 42 days, a pH greater than 6.98 following storage for 42 days, an ATP content greater than 86 percent of original ATP content at 21 days of storage, an ATP content greater than 60 percent of original ATP content at 42 days of storage, a 2,3-biphosphoglycerate (BPG) content greater than 44 percent of original BPG content at 21 days of storage, a BPG content greater than 10 percent of original BPG content at 42 days of storage, a plasma potassium concentration less than 21 mmol/L at 21 days of storage, a plasma potassium concentration less than 45 mmol/L at 42 days of storage, a plasma hemoglobin concentration less than 191 ng/L at 21 days of storage, a plasma hemoglobin concentration less than 386 ng/L at 42 days of storage, or any combination thereof.

In another embodiment, apparatus for storing red blood cells (RBC) comprises a storage container housing RBC in storage media and an electromagnetic stimulation zone to improve viability of the RBC.

In an embodiment, the RBC storage apparatus comprises an electric source to pass an electric current through the storage media, and the electric source can if desired include a controller to provide an amperage, voltage, wave form or a combination thereof corresponding to a cardiac cycle for the species of RBC. In embodiments, the electric source provides direct current or alternating current. The electric source in embodiments can include a controller to pulse the current at a frequency from 0.001 to 10 Hz; and/or to provide the current at a voltage potential between from 1 to 1000 millivolts.

In another embodiment, the RBC storage apparatus can additionally or alternatively comprise a magnetic field generator to apply a magnetic field to the RBC. In embodiments, the magnetic field comprises a static magnetic field or an oscillating magnetic field. The magnetic field can be either a homogenous magnetic field or a heterogeneous magnetic field. In exemplary embodiments, the magnetic field is within a range of from about 0.5 to about 500 Gauss or within a range of from about 10 to about 100 Gauss. The magnetic field generator in one example comprises a controller to pulse the magnetic field at a frequency from 0.001 to 10 Hz.

In the RBC storage apparatus according to one embodiment, the electromagnetic stimulation zone is disposed to apply the electromagnetic stimulation to the RBC just prior to or during transfusion into the recipient.

In one embodiment, the RBC storage media comprises whole blood and in a further embodiment comprises preservative solution such as, for example, citrate-potassium-dextrose solution (CPD) or citrate-potassium-dextrose-adenine solution (CPDA).

In one embodiment, the RBC storage apparatus comprises RBC separated from whole blood, and in a further embodiment comprises preservative solution such as, for example, adenine-saline solution (AS).

In an additional or alternative embodiment of the apparatus, the storage media can further comprise an added anticoagulant, pH buffer, nutrient, preservative, pathogen inactivator or combination thereof.

In an additional or alternative embodiment of the apparatus, the storage media further comprises an added rejuvenation solution.

The RBC storage apparatus in one embodiment can further comprise a gas exchange zone to add to the RBC or remove from the RBC oxygen, carbon dioxide or a combination thereof. In another embodiment, the RBC storage apparatus can further comprise a dialysis zone to remove waste products from the RBC.

In an additional or alternative embodiment, the RBC storage apparatus can comprise a shear zone to agitate the storage media comprising the RBC. In an embodiment, the RBC storage apparatus can include a pump to pump the storage media through an RBC flow circuit. For example, the RBC flow circuit can include the zone of electromagnetic stimulation.

In additional or alternative embodiments of the apparatus, a temperature control circuit is provided to maintain the temperature of the RBC, for example, between 1 and 6° C., or between about 30 and about 40° C.

In one embodiment, the electrical current or magnetic field applied to the RBC corresponds to the current or field applied to blood by the heart, either in a healthy heart or in the specific transfusion recipient, for example, a frequency and duration within 50% (i.e., 0.5 to 1.5 times the natural frequency or duration) or within 25% (i.e., 0.75 to 1.25 times the natural frequency or duration) of the electrical currents or fields ordinarily applied to blood from the atrioventricular node as it passes through the heart. In another embodiment, the strength of the current or field applied to the RBC is greater than that naturally applied in the right or left ventricle or right or left atrium, for example, 25, 50 or 100% greater, or from about twice to about 10 times greater, but not too great as to damage or injure the RBC, e.g. to avoid rouleaux. In one embodiment, the stored blood is periodically or continuously electrified or magnetized with a current and/or field effective to extend the life of the stored RBC. In other embodiments, the current or field is applied periodically to preserve the RBC, for example, from 1 to 5 seconds every 1 to 60 minutes or every 2 to 10 minutes, or from 30 seconds to 2 or 5 minutes every 1 to 12 hours, or for any duration and periodicity effective to improve the preservation and/or quality of the stored RBC. In an embodiment the electrical current and/or field are effective to inhibit charge depletion of the surface of the RBC, and in a further embodiment the electrical current and/or field are effective to maintain the magnetization levels of the heme irons in the RBC.

Thus, a patient can bank blood for autologous transfusion further in advance of surgery than is possible with conventional blood storage techniques, allowing the patient to fully recover from the blood loss. Further, the banked blood can be stored with a greater level of preservation or quality, which in one embodiment can be seen in the maintenance of uniform polarity of the RBC external surfaces. In other embodiments, the RBC have improved parameters indicative of viability, relative to conventional blood storage and preservation techniques, e.g., an increased proportion of viable cells (as determined 24 hours post transfusion) following storage, less pH loss or variation following storage, a greater ATP content greater relative to the original ATP content at collection, a greater 2,3-biphosphoglycerate (BPG) content relative to the original BPG content at storage, a lower plasma potassium concentration, a lower plasma hemoglobin concentration, or any combination thereof.

In one embodiment, the electromagnetic stimulation is applied to the blood as it is being transfused into the recipient, or just prior to transfusion, or for a period of time prior to transfusion to improve the RBC viability, e.g., for 6 to 24 hours prior to transfusion. For example, the current or field can be supplied to the transfusion container via electrodes and/or an external charging coil, which is activated during the transfusion, or in an embodiment before the transfusion for a duration effective to improve the external or surface polarity of the RBC, for example, 5 to 10 minutes. Where the RBC are treated for a sufficient duration prior to transfusion, the treatment can be continued at the same or a different, higher or lower current or field strength.

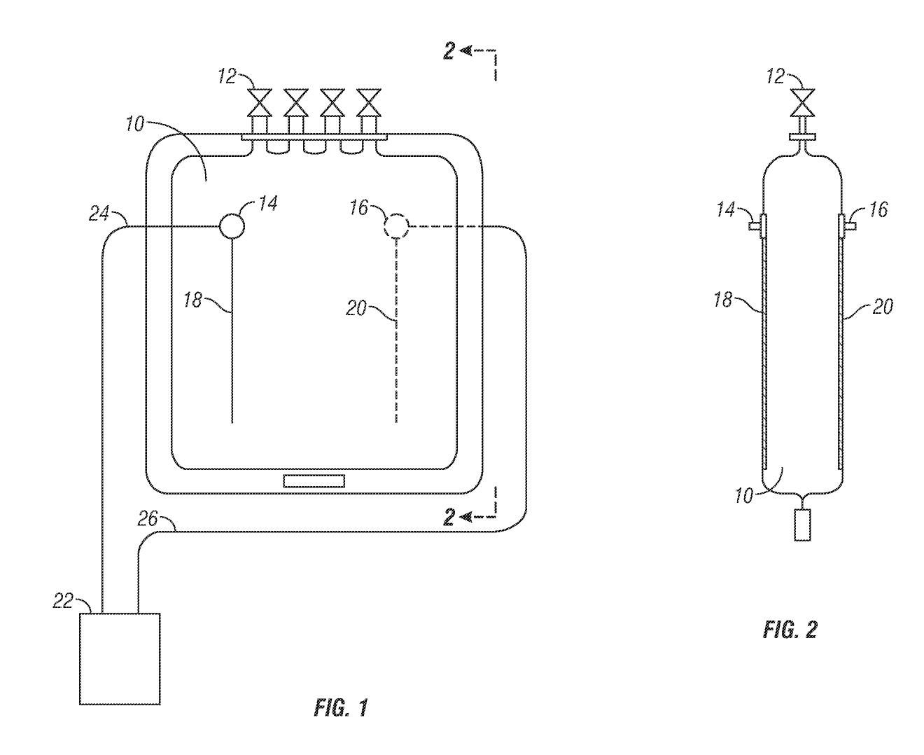

FIGS. 1 and 2 show an embodiment of blood storage in a bag 10 or other container having one or more ports 12 for filling or removing blood from the container 10. The container 10 is provided with a pair of electrodes 14, 16, which can include respective internal portions 18, 20 in electrical contact with the blood stored in the bag 10. For example, the internal portions 18, 20 can be flexible conductive wires adhered to or embedded at an inner surface of a wall of the blood bag 10. In another embodiment, the electrodes 14, 16 can be electrically connected via a biologically compatible wire mesh or wool within the bag 10 or other storage container. The internal electrodes 18, 20 can be positioned in an embodiment on opposite sides or ends of the container 10 to provide a relatively even current to the stored blood. An electrical current can be supplied from the controller 22 via conductors 24, 26 to the electrodes 14, 16 in electrical communication with the blood. In one embodiment, the electrical current is effective to inhibit rouleaux aggregation and/or induce rouleaux disaggregation in the stored RBC.

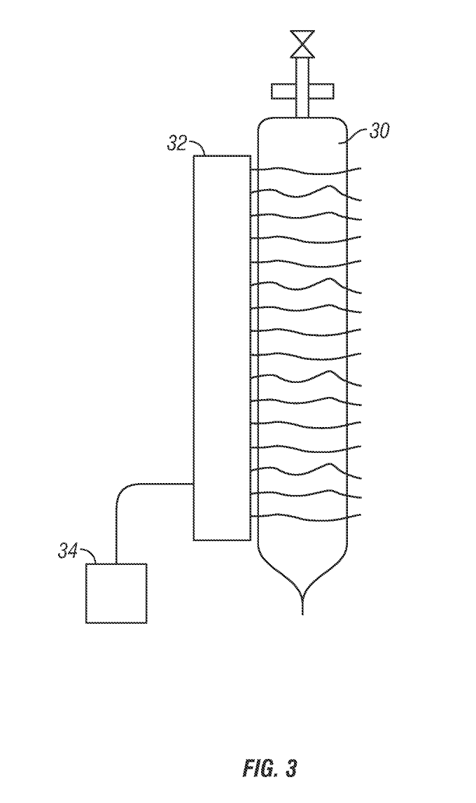

FIG. 3 shows an embodiment of blood storage in a bag 30 or other magnetically transparent container with wireless electromagnetic charge stimulation. Electromagnetic waves are pulsed through the bag 30 from an electromagnetic wave generator 32 adjacent to the bag 30. A controller 34 can be used to set the desired frequency and amplitude of the electromagnetic waves. In an embodiment, the magnetic field is within a range of from about 0.5 to about 500 Gauss, or within a range of from about 10 to about 100 Gauss. In an embodiment, the magnetic field is pulsed at a frequency from 0.001 to 10 Hz. Lower or higher intensity fields may also be used, for example, the magnetic field strength can range from greater than about 0.004, 1, 1.2, 10, 50, 100, 1000, 2500, 5000, or 10,000 Gauss or more. As examples of devices that may be suitably used as generator 32, non-limiting mention is made of degaussers commonly used to erase or scramble information stored on magnetic media, such as the Geneva PF-211 and PF-215 degaussers, which have degaussing electromagnetic field strengths of approximately 2300 and 2800 gauss, respectively, as well as the Bemer 3000 Mat or Bemer 300 Intensive Applicator supplied by Bemer USA, LLC. Of course, one of ordinary skill would understand that other devices producing a magnetic signal of sufficient field strength may also be used. In one embodiment, the electromagnetic field is effective to inhibit rouleaux aggregation and/or induce rouleaux disaggregation in the stored RBC.

The blood storage bags 10, 30 shown in FIGS. 1-3 can be stored in a temperature-controlled environment, e.g., a refrigerator or warming box. In one embodiment, the blood bags 10, 30 are stored in a refrigerator at 1 to 6° C. as is conventional. In another embodiment, the blood is maintained during storage at the normal body temperature of the animal from which it is taken, e.g., ±0.5° C., ±1° C., ±3° C., ±5° C., or ±10° C. In another embodiment, the blood storage bag or other container is maintained at human biological temperatures, e.g., from about 30° C. to about 42° C., or from about 30° C. to about 40° C., or from about 34° C. to about 40° C., or from about 35° C. to about 39° C., or about 37° C.±0.5° C. or ±1° C., or the like. Maintaining the temperature of the blood at approximately the biological temperature helps maintain the electromagnetic characteristics of the blood, e.g., it is known that dielectric characteristics of water change dramatically with 10° C. or 20° C. temperature changes. It is expected that the white blood cells initially present in whole blood will stop or inhibit bacterial growth during the initial storage conditions at normal biological temperatures, and then the bag or container will maintain sterility by preventing contamination from outside the bag or container membrane, which is preferably biologically impermeable. Alternatively or additionally, the blood or RBCs may be prepared for storage by applying pathogen inactivators and/or pathogen inactivation techniques as mentioned above.

In one embodiment, the blood bags 10, 30 shown in FIGS. 1-3 can be stored on a rocker or vibrator to provide constant or periodic motion to inhibit settling and/or aggregation of the RBC.

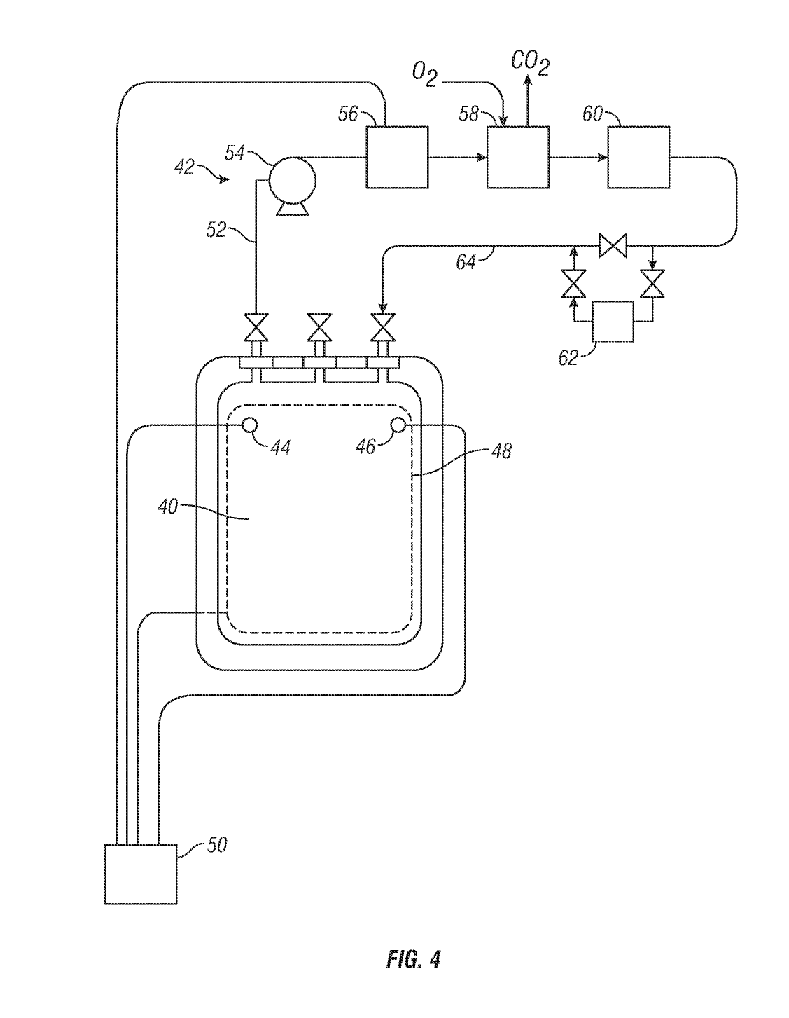

In another embodiment, as illustrated in FIG. 4, the storage device comprises at least one blood bag or container 40 and a flow circulation circuit 42. The blood bag 40 can be provided in one embodiment with electrodes 44, 46 and/or electromagnetic field generator 48 operated by controller 50 for electromagnetic stimulation in the bag 40 as described above in reference to FIGS. 1-3. The blood bag 40 can additionally or alternatively be stored on a rocker for additional agitation and/or in a temperature controlled room or larger container.

The flow circulation circuit 42 comprises tubing 52 or other flow conduit and at least one pump 54 to continuously or periodically circulate the blood during storage. In an embodiment, the pump 54 comprises a magnetically shielded flow path in order to avoid or minimize exposure of the RBC to magnetic fields employed in the pump 54, especially static magnetic fields. Magnetically shielded pumps are disclosed in my copending applications U.S. Ser. No. 12/433,566 filed Apr. 30, 2009, U.S. 61/409,838 filed Nov. 3, 2010, and U.S. 61/415,561 filed Nov. 19, 2010, which are hereby fully incorporated herein by reference. The pump 54 preferably provides pressures similar to those in the cardiovascular system of the animal from which the blood is taken, e.g., 8 to 21.3 or 32 kPa (60 to 160 or 240 mm Hg) in the case of human blood, to simulate biological conditions and avoid damaging the RBC by excessive fluid pressure.

The flow circuit 42 may include one or more of an electromagnetic stimulation unit 56, respiration unit 58, dialysis unit 60, or any combination thereof. The magnetic stimulation unit can include electrodes to apply a current to blood flowing through the unit 56, an electromagnetic field generator to apply a magnetic field to the blood flowing through the unit 56, or both. In an embodiment, the electromagnetic stimulation unit 56 is integrated with the pump 54 to electromagnetically stimulate the blood in the blood flow path through the pump 54/unit 56, for example, wherein the magnetic field(s) in the stator and/or rotor of the pump 54 also function to provide the appropriate electromagnetic stimulation of the RBC.

In one embodiment, the electromagnetic stimulation unit 56 can provide small parallel flow channels with a diameter on the same order of magnitude as that of an RBC or capillary in the animal from which the blood was obtained, e.g. within 100 to 200%, preferably from 105 to 150% of the mean RBC diameter, or from 50 to 200% of the mean capillary diameter. For example, the simulated capillaries can have a cross sectional diameter of 5 to 20 microns or 8 to 15 microns. If desired, unit 56 can include appropriate supply and return manifolds to distribute the RBC flow through a plurality of the microchannels. The channels can be formed, for example, by placing grooves in a face of an inert plastic plate, sheet, film or block or other suitable material, and then securing the face to another face which can also be grooved. Where the grooves are semicircular in cross section and match with a similar groove in the opposite face, a circular channel will be formed; or where the opposite face is flat or planar the channel will be semicircular. Other shapes may be used, but circular cross sections matching the animal's capillary size and configuration are preferred. The number of channels should be sufficient to provide the total desired flow area, e.g., within 50 to 200% of the total cross sectional flow area in the tubing 52, 64. The length of the channels is not critical, although in general they are as short as possible to minimize pressure drop and hydraulic damage of the RBC as they “squeeze” through the capillary-mimicking channels, e.g., 0.5 to 5 cm.

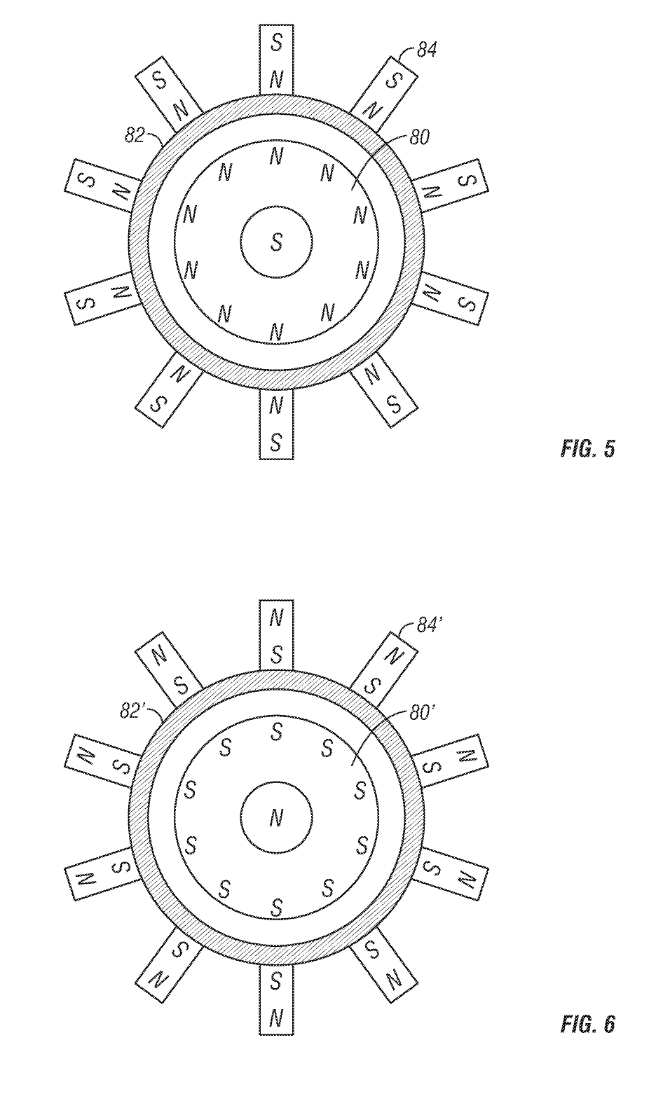

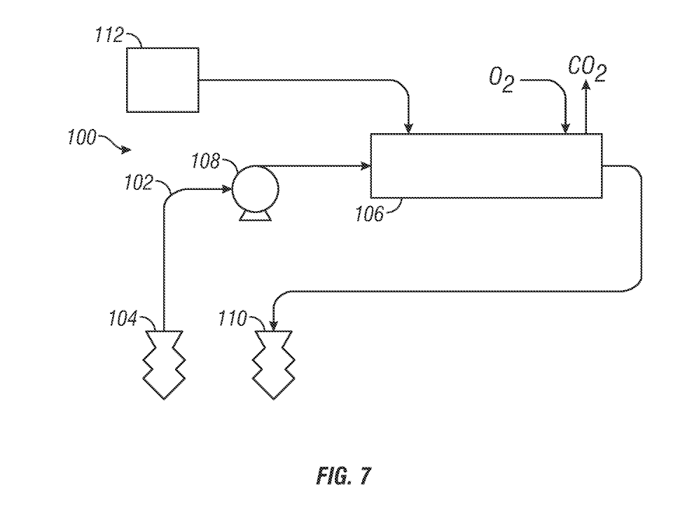

In one embodiment the electromagnetic stimulation unit 56 also includes at least one electromagnetic field generator, which can be a degaussing (alternating) magnetic field, a static or step-pulsed deoxygenating field (magnetic north oriented toward the RBC or a cathodic electrical field), or an oxygenating field (magnetic south oriented toward the RBC or an anodic electrical field), or any combination thereof. FIG. 5, for example, shows an RBC 80, which is fully oxygenated so that it has a north external polarity, passing through a capillary tube 82 wherein an external field 84 around the capillary 82 has a like polarity that tends to repel the RBC 80 so that sticking to the surface of the capillary 82 is less likely. In FIG. 6, the RBC 80′ is at least slightly deoxygenated so that it has a south external polarity, and the field 84′ has a south inward polarity. In one embodiment, especially at surfaces in contact with oxygenated blood, the magnets can have a “mild” magnetic field strength which is similar to that at the surface of normal erythrocytes. In this embodiment, the idea is to control the red blood cells from sticking together or to the exposed surfaces of the machine, but in one embodiment the field strength should not be so great as to induce oxygen release from the erythrocytes. A mild magnetic field can be attenuated in one embodiment by providing a relatively large flow cross section so that the magnets exert only an extremely minor field at the centerline or axis of the flow passage. In one embodiment, the magnets are provided at the oxygenator membranes, which may also optionally be heparinized as is known in the art.

In one embodiment, a first generator is provided to simulate electrobiological intracapillary deoxygenation in tissue and a second generator is provided downstream in series to simulate electrobiological intracapillary oxygenation in the lungs. In one embodiment, oxygen can be supplied and/or taken off via a gas permeable membrane in contact with the RBC in the microchannels just described, in the downstream respiration unit 58, or in the storage bag 40. Additionally or alternatively, if desired, carbon dioxide can be supplied and/or taken off via the same or different gas permeable membranes.

In another embodiment, an electrical current similar in voltage and current to that normally supplied at the atrioventricular node can be applied through the blood to and away from the oxygenator. In one embodiment the mild current is applied from an upstream electrode, to an electrode adjacent the oxygenator; in another embodiment from a downstream electrode, to the oxygenator electrode; and in another embodiment, the current is applied from both of the upstream and downstream electrodes to the common oxygenator electrode. In one embodiment, the current from the upstream and/or downstream electrodes is pulsed in a pattern similar to that of the atrioventricular node, and in another embodiment, the downstream electrode is pulsed approximately 0.02 seconds after the upstream electrode, corresponding to the current flow and pattern of the in vivo current from the atrioventricular node to the blood flowing between the heart and to/from the pulmonary capillaries.

In one embodiment, the current, electromagnetic field or combination thereof generated in the unit 56 is effective to inhibit rouleaux aggregation and/or induce rouleaux disaggregation in the RBC.

The respiration unit 58 can be or include, for example, a gas exchange unit to maintain desired levels of respiration gases, e.g., a membrane oxygenator to add and/or remove carbon dioxide and/or oxygen, to maintain oxygenation and carbon dioxide levels. Membrane oxygenators are well known for use in extracorporeal membrane oxygenation (ECMO) devices. In one embodiment the respiration unit 58 can be integrated with the electromagnetic stimulation unit 56, e.g., to provide electromagnetic stimulation during or in conjunction with gas exchange. For example, the electromagnetic stimulation (type, magnitude, frequency, polarity) associated with oxygen uptake and/or carbon dioxide release can mimic that which is biologically present in the air sac capillaries in the lungs, or the electromagnetic stimulation (type, magnitude, frequency, polarity) associated with oxygen release and/or carbon dioxide uptake can mimic that found in the tissues or organs other than the lungs. In one embodiment, the respiration unit 58 can include subunits in series to release oxygen/absorb carbon dioxide in a first subunit and to absorb oxygen/release carbon dioxide in the second subunit, as described above. In an embodiment, the RBC in return line 64 and blood bag 40 are more or less fully oxygenated, e.g. an oximetry of 98-100%.

Dialysis unit 60 is optional and can be applied continuously to remove waste components formed by the biological activity of the RBC, especially at normal biological temperatures. Dialysis is a well known procedure for patient's with no or impaired renal function. If desired, the blood can be supplemented with nutrients such as glucose or a slow release source of glucose can be added at the initial collection or processing of the blood or RBC in preparation for storage.

If desired, a side stream processing unit 62 can be provided in the return tubing 64. The unit 62 can include any type of hematological processing or testing equipment, or provide a sampling port for withdrawing specimens for testing or analysis. In one embodiment, the unit 62 includes an organ perfusion unit for maintaining the viability of the organ for transplant.

In an embodiment, the storage device can comprise two of the storage bags 40 to store oxygenated-state RBC and deoxygenated-state RBC, respectively. The blood under storage can be alternatingly pumped between the two bags in a first cycle to deoxygenate the RBC and in a second cycle to oxygenate the blood. The oxygenation and deoxygenation cycles can be provided in separate lines which are continuously operated in opposite directions more or less maintaining a constant blood volume in each storage container, or alternatively, the blood flow can be reversed in batch operations wherein the processing is alternated between batches between oxygenation and deoxygenation cycles. In one embodiment, the blood within the storage system can be pumped at a space velocity from 50% to 200% of the biological space velocity, e.g., from about 1 volume per 30 seconds to 1 volume per 2 minutes.

By repeatedly cycling the RBC through oxygenating and deoxygenating steps with similar biological hydrodynamic conditions, the electromagnetic health of the RBC can be viably maintained in storage up to about the same period of time as the RBC survives in vivo, or longer. To the extent the viability of RBC in vivo is a function of hydrodynamic conditions (where bumping and friction slowly degrade the RBC), the viability in storage can theoretically be further improved relative to biological conditions by providing comparatively improved hydrodynamic conditions, i.e., less bumping or friction by providing smooth walls, large radii turns, gradual diameter changes, elimination of obstructions and tortuous flow paths, maintaining laminar flow conditions, etc.

FIG. 7 relates to another embodiment wherein a heart lung machine or ECMO-type unit 100 is designed using an oxygenating flow circuit 102 that closely mimics pulmonary electromagnetic conditions in the body. Cardiopulmonary bypass devices remove deoxygenated blood from a venous cannula 104, through a series of tubes made from inert elastomeric materials and a membrane oxygenator 106, via a peristaltic or centrifugal pump 108, and returns the oxygenated blood to the patient at an arterial or venous cannula 110. The erythrocytes can be exposed to various electromagnetic fields in the prior art cardiopulmonary bypass process, e.g. from the pump or adjacent wires or other conductors where an electrical current is present, and in some cases these electromagnetic fields can induce the wrong polarity (“contramagnetic”) or insufficient (“hypomagnetic”) or excessive (“hypermagnetic”) strength (collectively, “dysmagnetic”) relative to erythrocytes passing normally (“eumagnetic”) through the right atrium and ventricle, to the pulmonary capillaries and then to the left atrium and ventricle, so that the blood is not properly magnetized and/or not properly oxygenated. It is believed that the dysmagentic conditions in cardiopulmonary bypass can contribute to Rouleaux formation, “sticky” blood that accumulates on the tubing, membrane and pump impeller surfaces, and may also cause or contribute to postperfusion syndrome, hemolysis, capillary leak syndrome, blood clotting in the oxygenator, air embolism, microembolic events, and the like.

In one embodiment, the tubing in the flow circuit 102 of FIG. 7 is constructed as shown in FIG. 5 and/or FIG. 6 discussed above, i.e., with an array of magnets or magnetic field generator 84, 84′ at the tubing wall 82, 82′, or spaced from but sufficiently close to the wall 82, 84 to induce a magnetic field into the tubing. In the case of the tubing 82, 82′, which has a circular section, the array is radial; however, in the case of a flat surface such as at an oxygenation membrane, the array may be planar. The magnetic field 84, 84′ can be static or electromagnets and are aligned with ends of like polarity facing toward the fluid flowing through the flow passage 102 and transporting the erythrocytes 80, 80′.

The magnets 84, 84′ in one embodiment exert a magnetic field onto the red blood cells 80, 80′, preferably of a like polarity with respect to the surface polarity of the erythrocytes 80, 80′. In one embodiment, the dipole orientation of the magnets 84, 84′ is the same as that of the surface of the red blood cell 80, 80′ so that there is a repulsion of the red blood cell 80, 80′ away from the inner surface of the tubing wall 82, 82′. For example, in FIG. 5 where the erythrocytes 80 have a north external polarity, the magnets 84 can be oriented with the north poles facing into the wall 82 of the flow passage 102 to push the erythrocytes away from the wall 82 and thereby inhibit adhesion or clotting at the surface, thus facilitating prevention of the accumulation of any “sticky” blood cells. In an alternative embodiment, an oscillating magnetic field is applied to alternatingly attract and repel the RBC to inhibit adhesion.

In one embodiment, especially at surfaces in contact with oxygenated blood, the magnets 84 can have a “mild” magnetic field strength which is similar to that at the surface of normal erythrocytes. In this embodiment, the idea is to control the red blood cells 80 from sticking together or to the exposed surfaces of the machine, but the field strength should not be so great as to induce oxygen release from the erythrocytes. A mild magnetic field can be attenuated in one embodiment by providing a relatively large flow cross section, e.g., 1-25 mm inside diameter, so that the magnets 80 exert only an extremely minor field at the centerline or axis of the flow passage 102. In one embodiment, the magnets 84 are provided at the oxygenator membranes in unit 106, which may also optionally be heparinized as is known in the art.

In another embodiment with reference to FIG. 7, the oxygenator 106 can include an electromagnetic stimulator similar in design and function to the unit 56 discussed above in connection with FIG. 4, and controller 110 to provide the electromagnetic stimulation as desired, e.g., application of a magnetic field or electric current, preferably in coordination with the oxygenation function of the oxygenator 106. In one embodiment, an electrical current similar in voltage and current to that normally supplied at the atrioventricular node can be applied through the blood passing through the tubing 102 leading to and away from the oxygenator 106. For example, the electrodes can be electrically connected via a biologically compatible wire mesh or wool within the tubing 102 or oxygenator 106. In one embodiment the mild current is applied from an upstream electrode, e.g. adjacent the venous cannula 104, to an electrode adjacent the oxygenator; in another embodiment from a downstream electrode, e.g., adjacent the arterial cannula 110, to the oxygenator electrode; and in another embodiment, the current is applied from both of the upstream and downstream electrodes to the common oxygenator electrode. In one embodiment, the current from the upstream and/or downstream electrodes is pulsed in a pattern similar to that of the atrioventricular node, and in another embodiment, the downstream electrode is pulsed approximately 0.02 seconds after the upstream electrode, corresponding to the current flow and pattern of the in vivo current from the atrioventricular node to the blood flowing between the heart and to/from the pulmonary capillaries.

Accordingly, the invention provides the following embodiments:
1. A method of storing red blood cells (RBC) comprising electromagnetically stimulating the RBC to improve viability.
2. The RBC storage method of embodiment 1 comprising periodically or continuously passing an electric current through the stored RBC.
3. The RBC storage method of embodiment 2 wherein the electric has an amperage, voltage, wave form or a combination thereof corresponding to a cardiac cycle for the species of RBC.
4. The RBC storage method of embodiment 2 or 3 wherein the current is direct current.
5. The RBC storage method of embodiment 2 or 3 wherein the current is alternating current.
6. The RBC storage method of any one of embodiments 2-4 wherein the current is pulsed at a frequency from 0.001 to 10 Hz.
7. The RBC storage method of any one of embodiments 2-6 wherein the current is supplied at a voltage potential between from 1 to 1000 millivolts.
8. The RBC storage method of any one of embodiments 1-7 comprising periodically or continuously applying a magnetic field to the RBC.
9. The RBC storage method of embodiment 8 wherein the magnetic field comprises a static magnetic field.
10. The RBC storage method of embodiment 8 or 9 wherein the magnetic field comprises an oscillating magnetic field.
11. The RBC storage method of any one of embodiments 8-10 wherein the magnetic field comprises a homogenous magnetic field.
12. The RBC storage method of any one of embodiments 8-10 wherein the magnetic field comprises a heterogeneous magnetic field.
13. The RBC storage method of any one of embodiments 8-12 wherein the magnetic field is within a range of from about 0.5 to about 500 Gauss.
14. The RBC storage method of any one of embodiments 8-12 wherein the magnetic field is within a range of from about 10 to about 100 Gauss.
15. The RBC storage method of any one of embodiments 8-14 wherein the magnetic field is pulsed at a frequency from 0.001 to 10 Hz.
16. The RBC storage method of any one of embodiments 1-15 wherein the electromagnetic stimulation is applied to the RBC just prior to or during transfusion into the recipient.
17. The RBC storage method of any one of embodiments 1-16 wherein the RBC are stored in whole blood.
18. The RBC storage method of embodiment 17 comprising storing the RBC in the presence of citrate-potassium-dextrose solution (CPD) or citrate-potassium-dextrose-adenine solution (CPDA).
19. The RBC storage method of any one of embodiments 1-16 comprising separating the RBC from whole blood.
20. The RBC storage method of embodiment 19 comprising storing the RBC in the presence of adenine-saline solution (AS).
21. The RBC storage method of embodiment 17 or 19 comprising storing the RBC in the presence of an added anticoagulant, pH buffer, nutrient, preservative or combination thereof.
22. The RBC storage method of embodiment 17, 19 or 21 comprising contacting the RBC with a rejuvenation solution.
23. The RBC storage method of embodiment 17, 19, 21 or 22 comprising irradiating the RBC.
24. The RBC storage method of any one of embodiments 1-23 comprising gas exchanging the RBC to add or remove oxygen, carbon dioxide or a combination thereof.
25. The RBC storage method of any one of embodiments 1-24 comprising dialysis to remove waste products from the RBC.
26. The RBC storage method of any one of embodiments 1-25 comprising agitating a medium comprising the RBC.
27. The RBC storage method of any one of embodiments 1-26 comprising pumping a medium comprising the RBC.
28. The RBC storage method of embodiment 27 wherein the RBC medium is passed through a zone of electromagnetic stimulation.
29. The RBC storage method of any one of embodiments 1-28 comprising controlling the temperature of the RBC between 1 and 6° C.
30. The RBC storage method of any one of embodiments 1-28 comprising controlling the temperature of the RBC between about 30 and about 40° C.
31. The RBC storage method of any one of embodiments 1-30 comprising storing the RBC in whole blood, optionally with an additive, for a period of time in excess of 35 days.
32. The RBC storage method of any one of embodiments 1-30 comprising storing the RBC for a period of time in excess of 45 days.
33. The RBC storage method of any one of embodiments 1-32 wherein the RBC comprise more than 84% viable cells (as determined 24 hours post transfusion) following storage for 42 days, a pH greater than 6.98 following storage for 42 days, an ATP content greater than 86 percent of original ATP content at 21 days of storage, an ATP content greater than 60 percent of original ATP content at 42 days of storage, a 2,3-biphosphoglycerate (BPG) content greater than 44 percent of original BPG content at 21 days of storage, a BPG content greater than 10 percent of original BPG content at 42 days of storage, a plasma potassium concentration less than 21 mmol/L at 21 days of storage, a plasma potassium concentration less than 45 mmol/L at 42 days of storage, a plasma hemoglobin concentration less than 191 ng/L at 21 days of storage, a plasma hemoglobin concentration less than 386 ng/L at 42 days of storage, or any combination thereof.
34. Apparatus for storing red blood cells (RBC), comprising a storage container housing RBC in storage media and a zone of electromagnetic stimulation to improve viability of the RBC.
35. The RBC storage apparatus of embodiment 34 comprising an electric source to pass an electric current through the storage media.
36. The RBC storage apparatus of embodiment 35 wherein the electric source comprises a controller to provide an amperage, voltage, wave form or a combination thereof corresponding to a cardiac cycle for the species of RBC.
37. The RBC storage apparatus of embodiment 35 or 36 wherein the electric source provides direct current.
38. The RBC storage apparatus of embodiment 35 or 36 wherein the electric source provides alternating current.
39. The RBC storage apparatus of any one of embodiments 36-38 wherein the electric source comprises a controller to pulse the current at a frequency from 0.001 to 10 Hz.
40. The RBC storage apparatus of any one of embodiments 36-39 wherein the electric source comprises a controller to provide the current at a voltage potential between from 1 to 1000 millivolts.
41. The RBC storage apparatus of any one of embodiments 36-40 comprising a magnetic field generator to apply a magnetic field to the RBC.
42. The RBC storage apparatus of embodiment 41 wherein the magnetic field comprises a static magnetic field.
43. The RBC storage apparatus of embodiment 41 or 42 wherein the magnetic field comprises an oscillating magnetic field.
44. The RBC storage apparatus of any one of embodiments 41-43 wherein the magnetic field comprises a homogenous magnetic field.
45. The RBC storage apparatus of any one of embodiments 41-43 wherein the magnetic field comprises a heterogeneous magnetic field.
46. The RBC storage apparatus of any one of embodiments 41-45 wherein the magnetic field is within a range of from about 0.5 to about 500 Gauss.
47. The RBC storage apparatus of any one of embodiments 41-45 wherein the magnetic field is within a range of from about 10 to about 100 Gauss.
48. The RBC storage apparatus of any one of embodiments 41-47 wherein the magnetic field generator comprises a controller to pulse the magnetic field at a frequency from 0.001 to 10 Hz.
49. The RBC storage apparatus of any one of embodiments 41-45 wherein the electromagnetic stimulation zone is disposed to apply the electromagnetic stimulation to the RBC just prior to or during transfusion into the recipient.
50. The RBC storage apparatus of any one of embodiments 34-49 wherein the storage media comprises whole blood.
51. The RBC storage apparatus of embodiment 50 wherein the storage media further comprises citrate-potassium-dextrose solution (CPD) or citrate-potassium-dextrose-adenine solution (CPDA).
52. The RBC storage apparatus of any one of embodiments 34-49 comprising RBC separated from whole blood.
53. The RBC storage apparatus of embodiment 52 wherein the storage media further comprises adenine-saline solution (AS).
54. The RBC storage apparatus of embodiment 50 or 52 wherein the storage media further comprises an added anticoagulant, pH buffer, nutrient, preservative or combination thereof.
55. The RBC storage apparatus of embodiment 50, 52 or 54 wherein the storage media further comprises an added rejuvenation solution.
56. The RBC storage apparatus of any one of embodiments 34-55 further comprising a gas exchange zone to add to the RBC or remove from the RBC oxygen, carbon dioxide or a combination thereof.
57. The RBC storage apparatus of any one of embodiments 34-56 further comprising a dialysis zone to remove waste products from the RBC.
58. The RBC storage apparatus of any one of embodiments 34-57 comprising a shear zone to agitate the storage media comprising the RBC.
59. The RBC storage apparatus of any one of embodiments 34-58 comprising a pump to pump the storage media through an RBC flow circuit.
60. The RBC storage apparatus of embodiment 59 wherein the RBC flow circuit comprises the zone of electromagnetic stimulation.
61. The RBC storage apparatus of any one of embodiments 34-60 further comprising a temperature control circuit to maintain the temperature of the RBC between 1 and 6° C.
62. The RBC storage apparatus of any one of embodiments 34-60 further comprising a temperature control circuit to maintain the temperature of the RBC between about 30 and about 40° C.
63. An extracorporeal membrane oxygenator (ECMO), comprising a flow circuit, a pump and an oxygenator, wherein the flow circuit comprises a flow path for red blood cells (RBC) to pass through an externally applied magnetic field, wherein the externally applied magnetic field has a like polarity relative to an external surface polarity of the RBC to inhibit sticking of the RBC to a surface of the flow path.

All numbers expressing quantities of ingredients, reaction conditions, and so forth in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Also, where a range is given, even if the term “between” is used, the ranges defined include the stated endpoints.

For all patents, applications, or other reference cited herein, it should be understood that such documents are incorporated by reference in their entirety for all purposes, as well as for any specifically recited proposition. Where any conflict exists between a document incorporated by reference and the present application, this application will dominate.

While various embodiments have been illustrated and described above, it will be appreciated that various changes can be made therein without departing from the spirit and scope of the present disclosure. It is, therefore, intended that the scope of the present disclosure be determined from the following claims and equivalents thereof.

1-23. (canceled)

24. An apparatus for storing red blood cells (RBC), comprising:
a storage container housing RBC in storage media; and
a zone of electromagnetic stimulation to improve viability of the RBC.

25. (canceled)

26. The apparatus for storing red blood cells of claim 24, wherein the electromagnetic stimulation periodically or continuously passes an electric current through the red blood cells.

27. The apparatus for storing red blood cells of claim 24, wherein the electromagnetic stimulation periodically or continuously applies a magnetic field to the red blood cells.

28. The apparatus for storing red blood cells of claim 27, wherein the magnetic field comprises an oscillating magnetic field.

29. The apparatus for storing red blood cells of claim 24, wherein the red blood cells are stored in whole blood.

30. The apparatus for storing red blood cells of claim 24, wherein the red blood cells are stored in the presence of an added anticoagulant, pH buffer, nutrient, preservative, pathogen inactivator system, or a combination thereof.

31. The apparatus for storing red blood cells of claim 24, further comprising:
a gas exchanger for adding or removing oxygen, or carbon dioxide, or a combination thereof to, or from the red blood cells.

32. The apparatus for storing red blood cells of claim 24, further comprising a dialysis zone to remove waste products from the red blood cells.

33. The apparatus for storing red blood cells of claim 24, further comprising a shear zone for agitating the storage media comprising the red blood cells.

34. The apparatus for storing red blood cells of claim 24, further comprising a temperature control device for controlling the temperature of the red blood cells between about 1° C. and about 6° C.

35. The apparatus for storing red blood cells of claim 24, further comprising a temperature control device for controlling the temperature of the red blood cells between about 30° C. and about 40° C.
